(19) United States
(12) Patent Application Publication
(10) Pub. No.: US 2023/0220041 A1
(43) Pub. Date: Jul. 13, 2023

(54) REGENERATIVE CAR-T CELLS

(71) Applicant: CREATIVE MEDICAL TECHNOLOGIES, INC., Phoenix, AZ (US)

(72) Inventors: Thomas Ichim, San Diego, CA (US); Amit Patel, Salt Lake City, UT (US); Timothy Warbington, Phoenix, AZ (US)

(73) Assignee: CREATIVE MEDICAL TECHNOLOGIES, INC., Phoenix, AZ (US)

(21) Appl. No.: 18/082,857

(22) Filed: Dec. 16, 2022

Related U.S. Application Data
(60) Provisional application No. 63/297,883, filed on Jan. 10, 2022.

(51) Int. Cl. C07K 14/725; C07K 14/54; C07K 14/49; C07K 14/515; C07K 14/65; C07K 14/475; C07K 16/18; A61K 35/17

(52) U.S. Cl. CPC C07K 14/7051; C07K 14/5403; C07K 14/49; C07K 14/515; C07K 14/65; C07K 14/4753; C07K 16/18; A61K 35/17; C07K 2319/33; C07K 2319/02; C07K 2319/03; A61K 2039/5156

(57) ABSTRACT

A method of creating antigen-specific, chimeric antigen receptor (CAR) T cells capable of secreting regenerative growth factors upon activation of said CAR. In one embodiment said regenerative CAR-T cells possess a CAR capable of selectively recognizing damaged tissue. In one embodiment said CAR recognizes damage-associated molecular patterns (DAMPs) such as ATP, HMGB-1, matricryptins, cold-inducible RNA-binding protein, histones and mitochondrial DNA. Upon activation of said CAR said regenerative CAR-T cell is induced to produce one or more regenerative growth factors. In some embodiments the invention provides a suicide gene in said regenerative CAR-T cells in order to remove said cells after their therapeutic purpose is completed.

### CROSS-REFERENCE TO RELATED APPLICATIONS

This application claims the benefit of priority to U.S. Provisional Application No. 63/297,883, filed Jan. 10, 2022, which is incorporated herein by reference in its entirety.

### FIELD OF THE INVENTION

The teachings herein relate to regenerative CAR-T lymphocytes and methods of using the same for treating tissue damage and other disorders.

### BACKGROUND OF THE INVENTION

Chimeric Antigen Receptor (CAR)-T cell therapy represents an emerging therapy for cancer, particularly in the treatment of B and T-cell lymphomas. CAR-T cell therapy comprises the use of adoptive cell transfer (ACT), a process which employs a subject's own T-cells which are modified using recombinant DNA techniques to express synthetic T-cell receptor (“TCR”) termed a chimeric antigen receptor (or “CAR”) alter the innate tropism of the T-cell so as to direct the engineered T-cell bind to a target cell. A CAR is typically an engineered fusion polyprotein which provides a synthetic T-cell receptor such that when the CAR contacts the ligand to which it is engineered to interact, the CAR-T-cell becomes activated. The chimeric antigen receptor is typically a single polypeptide comprising multiple functional domains, typically a targeting ectodomain that is expressed on the outer surface of a T-cell transformed with an expression vector encoding the CAR. The CAR further comprises a transmembrane domain that spans the cell membrane and an intracytoplasmic endodomain which mediates chemical reactions that provide intracellular signaling upon binding of the ectodomain to its target. For example, the ectodomain of the CAR may be specific for a known antigen present on a target cell. Frequently, the CAR is engineered to bind to a marker expressed on the surface of a neoplastic cell.

It is commonly known In the typical practice of CAR-T cell therapy, T-cells are isolated from a subject by apherisis and genetically altered to express CARs by transfecting the isolated T-cells ex vivo with a recombinant vector encoding a CAR resulting in a population of recombinantly modified CAR-T cells. CAR-T cells are often generated using patient-derived memory CD8+ T-cells recombinantly modified to express the CAR. Following ex vivo amplification, the CAR-T cells are typically infused back into the patient where the CAR-T cells circulate until the ectodomain of the CAR encounters its target binding ligand resulting in selective immune response to the target cell population.

In addition to the ability of CAR-T to selectively induce killing or damage to target cells such as tumor cells, there has been little utilization of antigen-specific ability of these cells to recognize and manipulate actions of other cells.

The current patent application discloses the utilization of CAR-T cells as a means of selectively modulating cells, especially in the context of regeneration.

In addition to T cells, the invention teaches generation of cells which are endowed with regenerative properties as result of CAR activation. In some embodiments, the population of immune cells is autologous to the recipient. In some embodiments, the population of immune cells is allogeneic to the recipient. In some embodiments, the immune cells is one or more of B cells, T cells, innate lymphoid cells, natural killer cells, natural killer T cells, gamma delta T cells, macrophages, monocytes, dendritic cells, neutrophils, myeloid derived suppressor cells, hematopoietic stem cells, or mesenchymal stem cells. In some embodiments, the population of immune cells is one or more of B cells, T cells, innate lymphoid cells, natural killer cells, natural killer T cells, gamma delta T cells, T regulatory cells, macrophages, monocytes, dendritic cells, neutrophils, myeloid derived suppressor cells.

### SUMMARY

Preferred embodiments are directed to methods of creating a regenerative chimeric antigen receptor (CAR) T cell comprising a chimeric antigen receptor (CAR) molecule, said CAR molecule having an antigen binding domain that binds to a molecule associated with tissue injury, furthermore said CAR callable of inducing expression of a molecule possessing regenerative activity.

Preferred methods include embodiments wherein said cell possesses a transmembrane domain, a co-stimulatory signaling region, and optionally an intracellular signaling domain.

Preferred methods include embodiments wherein the intracellular signaling domain comprises a CD3 zeta (CD3.zeta.) signaling domain.

Preferred methods include embodiments wherein the costimulatory signaling region comprises the cytoplasmic domain of a costimulatory molecule is CD27.

Preferred methods include embodiments wherein the costimulatory signaling region comprises the cytoplasmic domain of a costimulatory molecule is CD28.

Preferred methods include embodiments wherein the costimulatory signaling region comprises the cytoplasmic domain of a costimulatory molecule is 4-1 BB.

Preferred methods include embodiments wherein the costimulatory signaling region comprises the cytoplasmic domain of a costimulatory molecule is OX40.

Preferred methods include embodiments wherein the costimulatory signaling region comprises the cytoplasmic domain of a costimulatory molecule is CD30.

Preferred methods include embodiments wherein the costimulatory signaling region comprises the cytoplasmic domain of a costimulatory molecule is CD40.

Preferred methods include embodiments wherein the costimulatory signaling region comprises the cytoplasmic domain of a costimulatory molecule is PD-1.

Preferred methods include embodiments wherein the costimulatory signaling region comprises the cytoplasmic domain of a costimulatory molecule is ICOS.

Preferred methods include embodiments wherein the costimulatory signaling region comprises the cytoplasmic domain of a costimulatory molecule is LFA-1.

Preferred methods include embodiments wherein the costimulatory signaling region comprises the cytoplasmic domain of a costimulatory molecule is CD2.

Preferred methods include embodiments wherein the costimulatory signaling region comprises the cytoplasmic domain of a costimulatory molecule is CD7.

Preferred methods include embodiments wherein the costimulatory signaling region comprises the cytoplasmic domain of a costimulatory molecule is LIGHT.

Preferred methods include embodiments wherein the costimulatory signaling region comprises the cytoplasmic domain of a costimulatory molecule is NKG2C.

Preferred methods include embodiments wherein the costimulatory signaling region comprises the cytoplasmic domain of a costimulatory molecule is NKG2D.

Preferred methods include embodiments wherein the costimulatory signaling region comprises the cytoplasmic domain of a costimulatory molecule is B7-H3.

Preferred methods include embodiments wherein the costimulatory signaling region comprises the cytoplasmic domain of a costimulatory molecule is CD83 ligand.

Preferred methods include embodiments wherein said danger associated molecule is a DAMP.

Preferred methods include embodiments wherein said danger associated molecule is membrane bound vimentin.

Preferred methods include embodiments wherein said danger associated molecule is membrane heat shock protein.

The method of claim 22, wherein said heat shock protein is selected from a group comprising of: a) hsp 10; b) hsp 20/30; c) hsp 40; d) hsp 60; e) hsp 70; f) hsp90; and g) hsp 100.

Preferred methods include embodiments wherein said danger associated molecule is membrane calreticulin.

Preferred methods include embodiments wherein said danger associated molecule is thrombin.

Preferred methods include embodiments wherein said danger associated molecule is troponin.

Preferred methods include embodiments wherein said danger associated molecule is tissue factor.

Preferred methods include embodiments wherein said danger associated molecule is extrinsic factor.

Preferred methods include embodiments wherein said danger associated molecule is a complement activator.

Preferred methods include embodiments wherein said molecule associated with regenerative activity is interleukin-3.

Preferred methods include embodiments wherein said molecule associated with regenerative activity is interleukin-4.

Preferred methods include embodiments wherein said molecule associated with regenerative activity is interleukin-5.

Preferred methods include embodiments wherein said molecule associated with regenerative activity is interleukin-10.

Preferred methods include embodiments wherein said molecule associated with regenerative activity is interleukin-13.

Preferred methods include embodiments wherein said molecule associated with regenerative activity is interleukin-20.

Preferred methods include embodiments wherein said molecule associated with regenerative activity is interleukin-35.

Preferred methods include embodiments wherein said molecule associated with regenerative activity is interleukin-37.

Preferred methods include embodiments wherein said molecule associated with regenerative activity is TGF-alpha.

Preferred methods include embodiments wherein said molecule associated with regenerative activity is TGF-beta.

Preferred methods include embodiments wherein said molecule associated with regenerative activity is EGF.

Preferred methods include embodiments wherein said molecule associated with regenerative activity is IGF-1

Preferred methods include embodiments wherein said molecule associated with regenerative activity is HGF-1.

Preferred methods include embodiments wherein said molecule associated with regenerative activity is VEGF.

Preferred methods include embodiments wherein said molecule associated with regenerative activity is PDGF.

Preferred methods include embodiments wherein said molecule associated with regenerative activity is PDGF-BB.

Preferred methods include embodiments wherein said molecule associated with regenerative activity is FGF-1.

Preferred methods include embodiments wherein said molecule associated with regenerative activity is FGF-2.

Preferred methods include embodiments wherein said molecule associated with regenerative activity is FGF-5.

Preferred methods include embodiments wherein said molecule associated with regenerative activity is FGF-7.

Preferred methods include embodiments wherein said molecule associated with regenerative activity is NGF.

Preferred methods include embodiments wherein said molecule associated with regenerative activity is KGF.

Preferred methods include embodiments wherein said molecule associated with regenerative activity is BDNF.

Preferred methods include embodiments wherein said molecule associated with regenerative activity is erythropoietin.

Preferred methods include embodiments wherein said molecule associated with regenerative activity is G-CSF.

Preferred methods include embodiments wherein said molecule associated with regenerative activity is M-CSF.

Preferred methods include embodiments wherein said molecule associated with regenerative activity is GM-CSF.

Preferred methods include embodiments wherein said molecule associated with regenerative activity is LIF.

Preferred methods include embodiments wherein said molecule associated with regenerative activity is TPO.

Preferred methods include embodiments wherein said molecule associated with regenerative activity is GDF.

Preferred methods include embodiments wherein said molecule associated with regenerative activity is SDF-1.

Preferred methods include embodiments wherein said molecule associated with regenerative activity is neurotrophin.

Preferred methods include embodiments wherein said molecule associated with regenerative activity is interleukin-11.

Preferred methods include embodiments wherein said molecule associated with regenerative activity is CTGF.

Preferred methods include embodiments wherein said molecule associated with regenerative activity is activin.

Preferred methods include embodiments wherein said molecule associated with regenerative activity is Galectin 1-15.

Preferred methods include embodiments wherein said molecule associated with regenerative activity is epigen.

Preferred methods include embodiments wherein said molecule associated with regenerative activity is melanocyte stimulating factor.

Preferred methods include embodiments wherein said molecule associated with regenerative activity is MIP-1 alpha.

Preferred methods include embodiments wherein said molecule associated with regenerative activity is MIP-1 beta.

Preferred methods include embodiments wherein said molecule associated with regenerative activity is GDF-11.

Preferred methods include embodiments wherein said T cell is modified to possess reduced immunogenicity.

Preferred methods include embodiments wherein said reduction in immunogenicity is accomplished by deletion or silencing of beta 2 microglobulin.

Preferred methods include embodiments wherein said reduction in immunogenicity is accomplished by deletion or silencing of HLA I genes.

Preferred methods include embodiments wherein said reduction in immunogenicity is accomplished by deletion or silencing of HLA II genes.

Preferred methods include embodiments wherein said reduction in immunogenicity is accomplished by deletion or silencing of the CD40 gene.

Preferred methods include embodiments wherein said reduction in immunogenicity is accomplished by deletion or silencing of the CD80 gene.

Preferred methods include embodiments wherein said reduction in immunogenicity is accomplished by deletion or silencing of the CD86 gene.

Preferred methods include embodiments wherein said reduction in immunogenicity is accomplished by deletion or silencing of the IL-12 gene.

Preferred methods include embodiments wherein said reduction in immunogenicity is accomplished by deletion or silencing of the interferon alpha gene.

Preferred methods include embodiments wherein said reduction in immunogenicity is accomplished by deletion or silencing of the interferon beta gene.

Preferred methods include embodiments wherein said reduction in immunogenicity is accomplished by deletion or silencing of the interferon gamma gene.

Preferred methods include embodiments wherein said reduction in immunogenicity is accomplished by deletion or silencing of the interleukin-2 gene.

Preferred methods include embodiments wherein said reduction in immunogenicity is accomplished by deletion or silencing of the interleukin-6 gene.

Preferred methods include embodiments wherein said reduction in immunogenicity is accomplished by deletion or silencing of the interleukin-7 gene.

Preferred methods include embodiments wherein said reduction in immunogenicity is accomplished by deletion or silencing of the interleukin-11 gene.

Preferred methods include embodiments wherein said reduction in immunogenicity is accomplished by deletion or silencing of the interleukin-15 gene.

Preferred methods include embodiments wherein said reduction in immunogenicity is accomplished by deletion or silencing of the interleukin-16 gene.

Preferred methods include embodiments wherein said reduction in immunogenicity is accomplished by deletion or silencing of the interleukin-17 gene.

Preferred methods include embodiments wherein said reduction in immunogenicity is accomplished by deletion or silencing of the interleukin-18 gene.

Preferred methods include embodiments wherein said reduction in immunogenicity is accomplished by deletion or silencing of the interleukin-21 gene.

Preferred methods include embodiments wherein said reduction in immunogenicity is accomplished by deletion or silencing of the interleukin-22 gene.

Preferred methods include embodiments wherein said reduction in immunogenicity is accomplished by deletion or silencing of the interleukin-23 gene.

Preferred methods include embodiments wherein said reduction in immunogenicity is accomplished by deletion or silencing of the interleukin-27 gene.

Preferred methods include embodiments wherein said reduction in immunogenicity is accomplished by deletion or silencing of the interleukin-33 gene.

Preferred methods include embodiments wherein said T cell is engineered with an inducible suicide gene.

Preferred methods include embodiments wherein said T cell is manipulated to possess enhanced antiapoptotic activities.

Preferred methods include embodiments wherein said anti-apoptotic properties are mediated by enhancement of bcl-2 expression.

Preferred methods include embodiments wherein said anti-apoptotic properties are mediated by enhancement of bcl-xL expression.

Preferred methods include embodiments wherein said anti-apoptotic properties are mediated by enhancement of IAP-1 expression.

Preferred methods include embodiments wherein said anti-apoptotic properties are mediated by enhancement of IAP-2 expression.

Preferred methods include embodiments wherein said anti-apoptotic properties are mediated by enhancement of IAP-3 expression.

Preferred methods include embodiments wherein said anti-apoptotic properties are mediated by enhancement of IAP-4 expression.

Preferred methods include embodiments wherein said anti-apoptotic properties are mediated by enhancement of livin expression.

Preferred methods include embodiments wherein said anti-apoptotic properties are mediated by enhancement of survivin expression.

Preferred methods include embodiments wherein said anti-apoptotic properties are mediated by enhancement of interleukin-2 expression.

Preferred methods include embodiments wherein said anti-apoptotic properties are mediated by enhancement of interleukin-7 expression.

Preferred methods include embodiments wherein said anti-apoptotic properties are mediated by enhancement of PKC-zeta expression.

### DETAILED DESCRIPTION OF THE INVENTION

In one embodiment of the invention, CAR-T cells are generated to recognize cells undergoing cellular stress. Said CAR-T cells may be conventional T cells, or in other embodiments may be T regulatory cell.

“Allogeneic,” as used herein, refers to cells of the same species that differ genetically from cells of a host.

“Autologous,” as used herein, refers to cells derived from the same subject. The term “engraft” as used herein refers to the process of stem cell incorporation into a tissue of interest in vivo through contact with existing cells of the tissue.

“Approximately” or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Carrier” or diluent: As used herein, the terms “carrier” and “diluent” refers to a pharmaceutically acceptable (e.g., safe and non-toxic for administration to a human) carrier or diluting substance useful for the preparation of a pharmaceutical formulation. Exemplary diluents include sterile water, bacteriostatic water for injection (BWFI), a pH buffered solution (e.g. phosphate-buffered saline), sterile saline solution, Ringer's solution or dextrose solution.

As used herein, the terms “dosage form” and “unit dosage form” refer to a physically discrete unit of a therapeutic agent for the individual to be treated. Each unit contains a predetermined quantity of active material calculated to produce the desired therapeutic effect. It will be understood, however, that the total dosage of the composition will be decided by the attending physician within the scope of sound medical judgment.

A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, the therapeutic agent is administered continuously over a predetermined period. In some embodiments, the therapeutic agent is administered once a day (QD) or twice a day (BID).

By “does not detectably express ” means that expression of a protein or gene cannot be detected by standard methods. In the case of cell surface markers, expression can be measured by, e.g., flow cytometry, using a cut-off values as obtained from negative controls (i.e., cells known to lack the antigen of interest) or by isotype controls (i.e., measuring nonspecific binding of the antibody to the cell). Thus, a cell that “does not detectably express” a marker appears similar to the negative control for that marker. For gene expression, a gene “does not detectably express” if the presence of its mRNA cannot be visually detected on a standard agarose gel following standard PCR protocols. Conversely, a cell “expresses” the protein or gene if it can be detected by the same method.

The term “culture expanded population ” means a population of cells whose numbers have been increased by cell division in vitro. This term may apply to stem cell populations and non-stem cell populations alike.

The term “passaging” refers to the process of transferring a portion of cells from one culture vessel into a new culture vessel.

The term “cryopreserve” refers to preserving cells for long term storage in a cryoprotectant at low temperature.

The term “master cell bank ” refers to a collection of cryopreserved cells. Such a cell bank may comprise stem cells, non-stem cells, and/or a mixture of stem cells and non-stem cells.

For the purposes of promoting an understanding of the principles of the novel technology, reference will now be made to the preferred embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates are within the scope of this disclosure and the claims.

In some embodiments the CAR-T are utilized in a manner in which a regenerative gene is activated in response to a danger signal. For example, cells expressing calreticulin on their membranes are believed to be subjected to stress. Membrane expression of calreticulin is considered a sign of cellular stress [1-8]. Calreticulin was initially identified as the major Ca.sup.2+-storage protein in the sarcoplasmic reticulum of skeletal muscle. Subsequent work has revealed that the protein can also be detected in the endoplasmic reticulum of non-muscle tissues. Calreticulin has been considered to be a resident protein of the endoplasmic reticulum of a cell, where it is thought to behave as a calcium binding protein due to its high capacity calcium binding properties. Calreticulin possesses many diverse functional domains such as high affinity, low capacity- and low affinity, high capacity-Ca.sup.2+-binding sites, a C-terminal KDEL endoplasmic reticulum retention signal, and a nuclear localization signal. Calreticulin is also present in the nucleus of a cell, and it has been shown to have a consensus nuclear localization sequence which is highly homologous to that of histone proteins. Calreticulin is involved in DNA binding by nuclear hormone receptor and nuclear hormone receptor mediated gene transcription. Calreticulin also plays a role in regulation of integrin activity. Calreticulin associates with the cytoplasmic domains of integrin subunits. Integrins are mediators of cell adhesion to extracellular ligands. They can transduce biochemical signals into and out of cells. The cytoplasmic domains of integrins interact with several structural and signalling proteins and participate in the regulation of cell shape, motility, growth and differentiation. The interaction between calreticulin and the cytoplasmic domains of integrin subunits can influence integrin-mediated cell adhesion to extracellular matrix.

During cardiac infarcts it is known that various immunogenic molecules are released. In some embodiments the invention teaches generation of CAR expressing T cells that selectively home to the area of infarct and upon activation secrete regenerative factors. Numerous regenerative factors are known and are chosen based on degree of injury and qualitative nature of injury. Agents demonstrated to benefit various types of cardiac conditions include VEGF [9], HGF-1, FGF-2 and Angiopoietin.

In some embodiments of the invention, stimulation of T regulatory cells in vivo is accomplished by administration of Aldesleukin (Proleukin, Novartis), which is a commercially available IL-2 licensed for the treatment of metastatic renal cell carcinoma in the UK. It is produced by recombinant DNA technology using an Escherichia coli strain, which contains a genetically engineered modification of the human IL-2 gene, and is administered either intravenously or subcutaneously (SC). Following short intervenous infusion, its pharmacokinetic profile is typified by high plasma concentrations, rapid distribution into the extravascular space and a rapid renal clearance. The recommended doses for continuous infusion and subcutaneous injection (as detailed in the Summary of Product Characteristics) are repeated cycles of 18×10⁶ IU per m² per 24 hours for 5 days and repeated doses of 18×10⁶ IU, respectively. Peak plasma levels are reached in 2-6 hours after SC administration, with bioavailability of aldesleukin ranging between 31% and 47%. The process of absorption and elimination of subcutaneous aldesleukin is described by a one-compartment model, with a 45 min absorption half-life and an elimination half-life of 3-5 hours [10]. Natural IL-2 was first identified in 1976 as a growth factor for T lymphocytes. It is produced by human cluster designation (CD) 4+ and some CD8+ T-cells and is synthesized mainly by activated T-cells, in particular CD4.sup.+ helper T cells. It stimulates the proliferation and differentiation of T cells, induces the generation of cytotoxic T lymphocytes (CTLs) and the differentiation of peripheral blood lymphocytes to cytotoxic cells and lymphokine-activated killer (LAK) cells, promotes cytokine and cytolytic molecule expression by T cells, facilit:ites the proliferation and differentiation of B-cells and the synthesis of immunoglobulin by B-cells, and stimulates the generation, proliferation and activation of natural killer (NK). IL-2 is known to play a central role in the generation of immune responses. In cancer clinical trials, high-dose recombinant IL-2 (e.g., IV bolus dose of 600,000 international units (IU)/kg every 8 hours for up to 14 doses) demonstrated antitumor activity in metastatic renal cell carcinoma (RCC) and metastatic melanoma. Accordingly, such high-dose IL-2 was approved for the treatment of metastatic RCC in Europe in 1989 and in the US in 1992. In 1998, approval was obtained to treat patients with metastatic melanoma. Recombinant human IL-2 (Aldesleukin) (Proleukin.™.-Novartis Inc. & Prometheus Labs Inc.) is currently approved by the United States Food and Drug Administration (US FDA). However, IL-2 has a dual function in the immune response in that it not only mediates expansion and activity of effector cells, but also is crucially involved in maintaining peripheral immune tolerance. A major mechanism underlying peripheral self-tolerance is IL-2 induced activation-induced cell death (AICD) in T cells. AICD is a process by which fully activated T cells undergo programmed cell death through engagement of cell surface-expressed death receptors such as CD95 (also known as Fas) or the TNF receptor. When antigen-activated T cells expressing a high-affinity IL-2 receptor (after previous exposure to IL-2) during proliferation are re-stimulated with antigen via the T cell receptor (TCR)/CD 3 complex, the expression of Fas ligand (FasL) and/or tumor necrosis factor (TNF) is induced, making the cells susceptible for Fas-mediated apoptosis. This process is IL-2 dependent and mediated via STATS. By the process of AICD in T lymphocytes tolerance can not only be established to self-antigens, but also to persistent antigens that are clearly not part of the host's makeup, such as tumor antigens.

In some embodiments, the CAR-T cell is capable of producing a regenerative signal upon activation is a gene element which encodes a molecule or series of molecules that are secreted and the gene element is activated by a promoter associated with activation of the population of immune cells. In some embodiments, the promoter is activated as a consequence of T cell receptor signal transduction when the population of immune cells is a T cell, as a consequence of B cell receptor signal transduction when the population of immune cells is a B cell, as a consequence of NK activator receptor when the population of immune cells is a NK cell, as a consequence of NKG2D when the population of immune cells is a NK cell, as a consequence activation of a receptor of a danger associated molecular pattern (DAMP), or as a consequence activation of a pathogen associated molecular pattern (PAMP). In some embodiments, the DAMP receptor is one or more of a toll like receptor (TLR), a receptor for advanced glycation end products (RAGE), a siglec receptor, a stimulator of interferon genes (STING), a retinoic acid-inducible gene I (RIG-I), a melanoma differentiation-associated gene 5 (MDA5), or a Toll-interleukin 1 receptor domain (TIR)-containing adapter molecule 1 (TICAM-1). In some embodiments, the PAMP receptor is one or more of a toll like receptor (TLR), a receptor for advanced glycation end products (RAGE), a siglec receptor, a stimulator of interferon genes (STING), retinoic acid-inducible gene I (RIG-I), a melanoma differentiation-associated gene 5 (MDA5),or a Toll-interleukin 1 receptor domain (TIR)-containing adapter molecule 1 (TICAM-1).

In some embodiments of the invention, CAR-T cells possess a bispecific CAR in order to be activated by a tissue specific signal as well as a danger signal. In some embodiments a CAR-T cell is generated which produces growth factors upon ligation of said bispecific CAR to a hepatic antigen such as alpha fetal protein, concurrently with an injury associated signal such as calreticulin. Generation of bispecific CAR-T cells has been described in the literature and is shown in examples such as the following incorporated by reference. In one example a tandem CAR that joins folate receptor beta (FRβ) and CD123 in the second generation retroviral vector to generate a bispecific tandem CAR (TanCAR-T cell) was created. It was shown that TanCAR FRβ-CD123 T cells displayed distinct binding to FRβ or CD123 expressing cells. They could lyse the leukaemia cell lines (66.1±11%) comparable to the single CAR-T cells against these determinants. TanCAR FRβ-CD123 T cells simultaneously engaged FRβ and CD123, which promoted T cell activation in targeting and lysis of the examined leukaemia cell lines. TanCAR-T cell significantly induced interferon gamma (IFNγ) and interleukin 2 (IL-2) production more than single CAR-T cells, which produced a synergistic enhancement of TanCAR FRβ-CD123 T cell function when dual antigens faced simultaneously. The authors concluded that dual-specific TanCAR FRβ-CD123 T cells showed therapeutic potential to improve AML control by coengaging FRβ and CD123 molecules in a robust, divalent immune system [11].

In some embodiments of the invention, T cells are manipulated to increase production of endogenous cytokines in addition to transfected cytokines and/or growth factors that are inducible upon stimulation of the CAR-T cells of the invention. For example, in some embodiments T cells are silenced or gene edited for co-inhibitory molecules. The manipulation of CAR-T cells to possess enhanced activation ability has been demonstrated in previous studies which are incorporated by reference. In one example, Hu et al. showed that activated CD8+ T cells from PD-1H deficient (KO) mice exhibited increased cell proliferation, cytokine production and anti-tumor activity in vitro. Adoptive transfer of PD-1H-KO CD8+ T cells resulted in the regression of established syngeneic mouse tumors. Similar results were obtained when PD-1H was ablated in T cells by CRISPR/Cas9-mediated gene silencing. Furthermore, ablation of PD-1H in CAR-T cells significantly improved their anti-tumor activity against human xenografts in vivo [12].

In some embodiments of the invention regenerative CAR-T are administered locally into the target organ. Local administration may include perfusion of organ and/or deliver under high pressure [13]. In one embodiment, for example in the situation of heart failure, cells may be administered retrograde via the coronary sinus as developed by Dr. Amit Patel [14-20].

In certain embodiments, the present disclosure provides recombinant vectors comprising a nucleic acid sequence encoding a CAR and an HGF-1 agent and methods of use thereof.

In certain embodiments, the present disclosure provides recombinant vectors comprising a nucleic acid sequence encoding a CAR and an FGF-1 agent and methods of use thereof.

In certain embodiments, the present disclosure provides recombinant vectors comprising a nucleic acid sequence encoding a CAR and an FGF-2 agent and methods of use thereof.

In certain embodiments, the present disclosure provides recombinant vectors comprising a nucleic acid sequence encoding a CAR and an FGF-5 agent and methods of use thereof.

In certain embodiments, the present disclosure provides recombinant vectors comprising a nucleic acid sequence encoding a CAR and an FGF-7 agent and methods of use thereof.

In certain embodiments, the present disclosure provides recombinant vectors comprising a nucleic acid sequence encoding a CAR and an VEGF-A agent and methods of use thereof.

In certain embodiments, the present disclosure provides recombinant vectors comprising a nucleic acid sequence encoding a CAR and an VEGF-B agent and methods of use thereof.

In certain embodiments, the present disclosure provides recombinant vectors comprising a nucleic acid sequence encoding a CAR and an angiopoietin agent and methods of use thereof.

In certain embodiments, the present disclosure provides recombinant vectors comprising a nucleic acid sequence encoding a CAR and an GM-CSF agent and methods of use thereof.

In certain embodiments, the present disclosure provides recombinant vectors comprising a nucleic acid sequence encoding a CAR and an G-CSF agent and methods of use thereof.

In certain embodiments, the present disclosure provides recombinant vectors comprising a nucleic acid sequence encoding a CAR and an M-CSF agent and methods of use thereof.

In certain embodiments, the present disclosure provides recombinant vectors comprising a nucleic acid sequence encoding a CAR and an IGF-1 agent and methods of use thereof.

In certain embodiments, the present disclosure provides recombinant vectors comprising a nucleic acid sequence encoding a CAR and an EGF agent and methods of use thereof.

In certain embodiments, the present disclosure provides recombinant vectors comprising a nucleic acid sequence encoding a CAR and an BDNF agent and methods of use thereof.

In certain embodiments, the present disclosure provides recombinant vectors comprising a nucleic acid sequence encoding a CAR and an CNTF agent and methods of use thereof.

In certain embodiments, the present disclosure provides recombinant vectors comprising a nucleic acid sequence encoding a CAR and an GDNF agent and methods of use thereof.

In certain embodiments, the present disclosure provides recombinant vectors comprising a nucleic acid sequence encoding a CAR and an neutrophin-1 agent and methods of use thereof.

In certain embodiments, the present disclosure provides recombinant vectors comprising a nucleic acid sequence encoding a CAR and a LIF agent and methods of use thereof.

In certain embodiments, the present disclosure provides recombinant vectors comprising a nucleic acid sequence encoding a CAR and an PDGF-BB agent and methods of use thereof.

In certain embodiments, the present disclosure provides recombinant vectors comprising a nucleic acid sequence encoding a CAR and an placental derived growth factor agent and methods of use thereof.

In certain embodiments, the present disclosure provides recombinant vectors comprising a nucleic acid sequence encoding a CAR and a GDNF agent and methods of use thereof.

Additional embodiments of the present disclosure contemplate methods of treating a subject having a degenerative disease, disorder or condition, comprising introducing to the subject a therapeutically effective plurality of cells genetically modified to express a) a chimeric antigen receptor, wherein the chimeric antigen receptor comprises at least one antigen-specific targeting region capable of binding to the target cell population, and b) a regenerative agent. In some embodiments, the chimeric antigen receptor and the regenerative agent are expressed by the same vector, while in other embodiments the chimeric antigen receptor and the regenerative agent are expressed by different vectors.

In particular embodiments, the therapeutically effective plurality of cells is transfected with a vector that expresses the regenerative agent in an amount sufficient to enhance proliferation and activation of endogenous progenitor cells. The vector may be, for example, a non-viral or a viral vector. The present disclosure also contemplates the use of any other means of expressing the regenerative agent. In particular embodiments, expression of the regenerative agent is modulated by an expression control element. In particular embodiments, the expression control element is a regulatable promoter. In particular embodiments, the expression control element is tissue specific promoter.

In the embodiments described above, the plurality of cells may be obtained from the subject and genetically modified ex vivo. According to some embodiments of the present disclosure, the plurality of cells is obtained from the subject by an aphaeretic process at treated with at least one regenerative agent following expansion and for a period of time prior to administration, the period of time being less than about 48 hours, less than about 36 hours, less than about 24 hours, less than about 18 hours, less than about 12 hours, less than about 6 hours, less than about 4 hours, less than about 2 hours, or less than about 1 hour prior to administration to the subject. cells in other embodiments.

Still further embodiments of the present disclosure contemplate methods of treating a subject having a degenerative-related disease, disorder or condition, comprising introducing to the subject a) a therapeutically effective first plurality of cells genetically modified to express a chimeric antigen receptor, wherein the chimeric antigen receptor comprises at least one antigen-specific targeting region capable of binding to the target cell population, and b) a therapeutically effective second plurality of cells genetically modified to express a regenerative agent.

In some embodiments, the regenerative CAR-T cell employed as described herein provides an intracellular signaling domain comprising an amino acid sequence derived from the cytoplasmic domain of CD27, CD28, CD137 CD278, CD134, Fc.epsilon.R1.gamma. and .beta. chains, MB1 (Ig.alpha.) chain, B29 (Ig.beta.) chain, the human CD3 zeta chain, CD3, a syk family tyrosine kinase, a src family tyrosine kinase, CD2, CD5 or CD28. In one embodiment, for CAR-T cell used in the practice of the method provides an intracellular signaling domain comprising an amino acid sequence derived from the cytoplasmic domain of CD28, CD137 (4-1BB), CD134 (0X40), Dap10, CD27, CD2, CD5, ICAM-1, LFA-1 (CD11a/CD18), Lck, TNFR-I, TNFR-II, Fas, CD30, and CD40. The foregoing method may be combined with the administration to the subject of one or more supplemental agents including chemotherapeutic agents, immune checkpoint modulators, IL-2 agents, IL-7 agents, IL-12 agents, IL-15 agents and IL-18 agents, in particular where the immune checkpoint modulators selected from the group consisting of PD1 modulators, PDL1 modulators, CTLA4 modulators, LAG-3 modulators, TIM-3 modulators, ICOS modulators, 0X40 modulators, cd-27 modulators, CD-137 modulators, HVEM modulators, CD28 modulators, CD226 modulators, GITR modulators, BTLA modulators, A2A modulators, IDO modulators and VISTA modulators.

Nucleic acid molecules encoding the regenerative agents are contemplated by the present disclosure, including their naturally-occurring and non-naturally occurring isoforms, allelic variants and splice variants. The present disclosure also encompasses nucleic acid sequences that vary in one or more bases from a naturally-occurring DNA sequence but still translate into an amino acid sequence that corresponds to a regenerative agent polypeptide due to degeneracy of the genetic code. Where a polypeptide is produced using recombinant techniques, the polypeptide can be produced as an intracellular protein or as a secreted protein, using any suitable construct and any suitable host cell, which can be a prokaryotic or eukaryotic cell, such as a bacterial (e.g., E. coli) or a yeast host cell, respectively. Other examples of eukaryotic cells that can be used as host cells include insect cells, mammalian cells, and/or plant cells. Where mammalian host cells are used, they can include human cells (e.g., HeLa, 293, H9 and Jurkat cells); mouse cells (e.g., NIH3T3, L cells, and C127 cells); primate cells (e.g., Cos 1, Cos 7 and CV1); and hamster cells (e.g., Chinese hamster ovary (CHO) cells).

For the practice of the invention, a variety of host-vector systems suitable for the expression of a polypeptide can be employed according to standard procedures known in the art. See, e.g., Sambrook, et al., (1989) Current Protocols in Molecular Biology Cold Spring Harbor Press, New York; and Ausubel, et al. (1995) Current Protocols in Molecular Biology, Eds. Wiley and Sons. Methods for introduction of genetic material into host cells include, for example, transformation, electroporation, conjugation, calcium phosphate methods and the like. The method for transfer can be selected so as to provide for stable expression of the introduced polypeptide-encoding nucleic acid. The polypeptide-encoding nucleic acid can be provided as an inheritable episomal element (e.g., a plasmid) or can be genomically integrated. A variety of appropriate vectors for use in production of a polypeptide of interest are commercially available. Vectors can provide for extrachromosomal maintenance in a host cell or can provide for integration into the host cell genome. The expression vector provides transcriptional and translational regulatory sequences and can provide for inducible or constitutive expression where the coding region is operably-linked under the transcriptional control of the transcriptional initiation region, and a transcriptional and translational termination region. In general, the transcriptional and translational regulatory sequences can include, but are not limited to, promoter sequences, ribosomal binding sites, transcriptional start and stop sequences, translational start and stop sequences, and enhancer or activator sequences. Promoters can be either constitutive or inducible, and can be a strong constitutive promoter (e.g., T7). Expression constructs generally have convenient restriction sites located near the promoter sequence to provide for the insertion of nucleic acid sequences encoding proteins of interest. A selectable marker operative in the expression host can be present to facilitate selection of cells containing the vector. Moreover, the expression construct can include additional elements. For example, the expression vector can have one or two replication systems, thus allowing it to be maintained in organisms, for example, in mammalian or insect cells for expression and in a prokaryotic host for cloning and amplification. In addition, the expression construct can contain a selectable marker gene to allow the selection of transformed host cells. Selectable genes are well known in the art and will vary with the host cell used.

It is known the art, and applicable to the current invention, isolation and purification of a protein can be accomplished according to methods known in the art. For example, a protein can be isolated from a lysate of cells genetically modified to express the protein constitutively and/or upon induction, or from a synthetic reaction mixture by immunoaffinity purification, which generally involves contacting the sample with an anti-protein antibody, washing to remove non-specifically bound material, and eluting the specifically bound protein. The isolated protein can be further purified by dialysis and other methods normally employed in protein purification. In one embodiment, the protein can be isolated using metal chelate chromatography methods. Proteins can contain modifications to facilitate isolation. The polypeptides can be prepared in substantially pure or isolated form (e.g., free from other polypeptides). The polypeptides can be present in a composition that is enriched for the polypeptide relative to other components that can be present (e.g., other polypeptides or other host cell components). For example, purified polypeptide can be provided such that the polypeptide is present in a composition that is substantially free of other expressed proteins, e.g., less than about 90%, less than about 60%, less than about 50%, less than about 40%, less than about 30%, less than about 20%, less than about 10%, less than about 5%, or less than about 1%. The regenerative polypeptide or protein can be generated using recombinant techniques to manipulate nucleic acids known in the art to provide constructs capable of encoding the polypeptide. It will be appreciated that, when provided a particular amino acid sequence, the ordinary skilled artisan will recognize a variety of different nucleic acid molecules encoding such amino acid sequence in view of her background and experience in, for example, molecular biology.

### REFERENCES

1. Meyer, B. A. and S. Doroudgar, ER Stress-Induced Secretion of Proteins and Their Extracellular Functions in the Heart. Cells, 2020. 9(9).
2. Stoner, M. W., et al., Calreticulin expression in human cardiac myocytes induces ER stress-associated apoptosis. Physiol Rep, 2020. 8(8): p. e14400.
3. Azuma, Y., et al., Biphasic Increases of Cell Surface Calreticulin Following Treatment with Mitoxantrone. Biol Pharm Bull, 2020. 43(10): p. 1595-1599.
4. Yang, P. M., et al., Sequential Interferon beta-Cisplatin Treatment Enhances the Surface Exposure of Calreticulin in Cancer Cells via an Interferon Regulatory Factor 1-Dependent Manner. Biomolecules, 2020. 10(4).
5. Han, Y. K., et al., Hypoxia induces immunogenic cell death of cancer cells by enhancing the exposure of cell surface calreticulin in an endoplasmic reticulum stress-dependent manner. Oncol Lett, 2019. 18(6): p. 6269-6274.
6. Joshi, R., et al., Role of calreticulin in biotic and abiotic stress signalling and tolerance mechanisms in plants. Gene, 2019. 714: p. 144004.
7. Pandya, U. M., et al., The Biophysical Interaction of the Danger-Associated Molecular Pattern (DAMP) Calreticulin with the Pattern-Associated Molecular Pattern (PAMP) Lipopolysaccharide. Int J Mol Sci, 2019. 20(2).
8. Zhao, D., et al., Cardiac-derived CTRP9 protects against myocardial ischemia/reperfusion injury via calreticulin-dependent inhibition of apoptosis. Cell Death Dis, 2018. 9(7): p. 723.
9. Rasanen, M., et al., VEGF-B Promotes Endocardium-Derived Coronary Vessel Development and Cardiac Regeneration. Circulation, 2021. 143(1): p. 65-77.
10. Zhao, T. X., et al., Low-dose interleukin-2 in patients with stable ischaemic heart disease and acute coronary syndromes (LILACS): protocol and study rationale for a randomised, double-blind, placebo-controlled, phase I/II clinical trial. BMJ Open, 2018. 8(9): p. e022452.
11. Ghamari, A., et al., Design and Production An Effective Bispecific Tandem Chimeric Antigen Receptor on T Cells against CD123 and Folate Receptor ss towards B-Acute Myeloid Leukaemia Blasts. Cell J, 2021. 23(6): p. 650-657.
12. Hu, L., et al., Ablation of T cell-associated PD-1H enhances functionality and promotes adoptive immunotherapy. JCI Insight, 2021.
13. Chai, L. F., et al., Regional Delivery of CAR-T Effectively Controls Tumor Growth in Colorectal Liver Metastasis Model. J Surg Res, 2021. 272: p. 37-50.
14. Rodenberg, E. J., et al., Catheter-based retrograde coronary sinus infusion is a practical delivery technique for introducing biological molecules into the cardiac system. Catheter Cardiovasc Interv, 2019. 94(5): p. 669-676.
15. Pleva, L., et al., Rationale and design of a prospective, randomised study of retrograde application of bone marrow aspirate concentrate (BMAC) through coronary sinus in patients with congestive heart failure of ischemic etiology (the RETRO study). BMC Cardiovasc Disord, 2019. 19(1): p. 32.
16. Yuniadi, Y., et al., Progenitor Hematopoietic Cells Implantation Improves Functional Capacity of End Stage Coronary Artery Disease Patients with Advanced Heart Failure. Cardiol Res Pract, 2016. 2016: p. 3942605.
17. Tuma, J., et al., RESCUE-HF Trial: Retrograde Delivery of Allogeneic Umbilical Cord Lining Subepithelial Cells in Patients With Heart Failure. Cell Transplant, 2016. 25(9): p. 1713-1721.
18. Patel, A. N., et al., REVIVE Trial: Retrograde Delivery of Autologous Bone Marrow in Patients With Heart Failure. Stem Cells Transl Med, 2015. 4(9): p. 1021-7.
19. Tuma, J., et al., Safety and feasibility of percutaneous retrograde coronary sinus delivery of autologous bone marrow mononuclear cell transplantation in patients with chronic refractory angina. J Transl Med, 2011. 9: p. 183.
20. George, J. C., et al., Transvenous intramyocardial cellular delivery increases retention in comparison to intracoronary delivery in a porcine model of acute myocardial infarction. J Interv Cardiol, 2008. 21(5): p. 424-31.

1. A method of creating a regenerative chimeric antigen receptor (CAR) T cell comprising a chimeric antigen receptor (CAR) molecule, said CAR molecule having an antigen binding domain that binds to a molecule associated with tissue injury, furthermore said CAR capable of inducing expression of a molecule possessing regenerative activity.

2. The method of claim 1, wherein said cell possesses a transmembrane domain, a co-stimulatory signaling region, and optionally an intracellular signaling domain.

3. The method of claim 1, wherein the intracellular signaling domain comprises a CD3 zeta (CD3.zeta.) signaling domain.

4. The method of claim 1, wherein the costimulatory signaling region comprises the cytoplasmic domain of a costimulatory molecule is selected from a group comprising of: a) CD28; b) 4-1 BB; c) OX40; d) CD30; e) interleukin-12; f) interleukin-15; g) CD40; h) PD-1; i) ICOS; j) LFA-1; k) CD2; 1) CD7; m) LIGHT; n) NKG2C; o) NKG2D; p) B7-H3; and q) CD83 ligand.

5. The method of claim 1, wherein said danger associated molecule is a DAMP.

6. The method of claim 1, wherein said danger associated molecule is membrane bound vimentin.

7. The method of claim 1, wherein said danger associated molecule is membrane heat shock protein.

8. The method of claim 7, wherein said heat shock protein is selected from a group comprising of: a) hsp 10; b) hsp 20/30; c) hsp 40; d) hsp 60; e) hsp 70; f) hsp90; and g) hsp 100.

9. The method of claim 1, wherein said danger associated molecule is membrane calreticulin.

10. The method of claim 1, wherein said danger associated molecule is thrombin.

11. The method of claim 1, wherein said danger associated molecule is troponin.

12. The method of claim 1, wherein said danger associated molecule is tissue factor.

13. The method of claim 1, wherein said danger associated molecule is extrinsic factor.

14. The method of claim 1, wherein said danger associated molecule is a complement activator.

15. The method of claim 1, wherein said molecule associated with regenerative activity is interleukin-3.

16. The method of claim 1, wherein said molecule associated with regenerative activity is VEGF.

17. The method of claim 1, wherein said molecule associated with regenerative activity is angiopoietin.

18. The method of claim 1, wherein said molecule associated with regenerative activity is IGF-1.

19. The method of claim 1, wherein said molecule associated with regenerative activity is placental growth factor.

20. The method of claim 1, wherein said molecule associated with regenerative activity is hepatocyte growth factor.
